(19) United States
(12) Patent Application Publication
(10) Pub. No.: US 2013/0190200 A1
(43) Pub. Date: Jul. 25, 2013

(54) SNP FOR PREDICTING THE SENSITIVITY TO ANTICANCER TARGETED THERAPEUTIC FORMULATION

(71) Applicants: Jin Cheon Kim, Seoul (KR); Yong Sung Kim, Daejeon (KR); Seon Young Kim, Daejeon (KR); Dong Hyung Cho, Seoul (KR)

(72) Inventors: Jin Cheon Kim, Seoul (KR); Yong Sung Kim, Daejeon (KR); Seon Young Kim, Daejeon (KR); Dong Hyung Cho, Seoul (KR)

(73) Assignees: Korea Research Institute of Bioscience and Biotechnology, Daejeon (KR); The ASAN Foundation, Seoul (KR)

(21) Appl. No.: 13/704,423

(22) PCT Filed: Dec. 8, 2010
(86) PCT No.: PCT/KR2010/008734

§ 371 Date: Feb. 25, 2013

(30) Foreign Application Priority Data
Jun. 15, 2010 (KR) 10-2010-0056279

(51) Int. Cl. C12Q 1/68

(52) U.S. Cl. CPC C12Q 1/6886
USPC 506/9; 536/24.33; 536/24.31; 506/16; 435/6.11

(57) ABSTRACT

The present invention relates to a single nucleotide polymorphism (SNP) for predicting the sensitivity to an anticancer targeted therapeutic formulation, a polynucleotide containing the same, and a method for predicting the sensitivity to an anticancer targeted therapeutic formulation. According to the present invention, it is possible to predict the sensitivity of each individual to a certain anticancer targeted therapeutic formulation, using a small amount of a sample taken from a patient and thus to select a most suitable targeted therapeutic formulation over the entire duration of treatment for the patient.

### CROSS-REFERENCE TO RELATED APPLICATIONS

This application is a national stage of PCT/KR2010/008734, filed on Dec. 8, 2010, which claims priority to Korean Patent Application No. 10-2010-0056279, filed Jun. 15, 2010.

### FIELD OF THE INVENTION

The present invention relates to a single nucleotide polymorphism (SNP) marker for predicting the sensitivity to an anticancer targeted drug, a polynucleotide containing the same, and a method for predicting the sensitivity to a specific anticancer targeted drug.

### DESCRIPTION OF THE BACKGROUND

Various genetic differences between individuals are most attributable to single nucleotide polymorphisms, and some genotypes exhibit functional differences in corresponding proteins and their metabolism, which is known to be susceptible to pharmacogenetic individual sensitivities (Huang and Ratain, C A Cancer J Clin 2009; 59:42-55).

Meanwhile, many anticancer drugs are used as effective therapeutic agents, but a new problem is the resistance of cancer cells (Tsuruo et al., 1985). The resistance to anticancer drugs is caused by various mechanisms in which cells exposed to drugs due to long-term use of anticancer drugs reduce the accumulation of drugs in cells (Shen et al., 1986; Shen et al., 2000; Gottesman et al., 2002), activate detoxification or degradation (Schuetz et al., 1999; Goto et al., 2000), or modify targeted proteins (Urasaki et al., 2001). These processes are the most significant obstacle in the treatment of cancers and closely related to treatment failures (Kang, 1996). Among various mechanisms involved in the resistance to drugs, multidrug resistance (MDR) plays an important role (Brooks et al., 2003). Multidrug resistance refers to a phenomenon that causes the resistance to various anticancer drugs with entirely different structures or functions as well as to anticancer drugs being used (Mehta et al., 1992; Gottesman, 2000). If a specific anticancer drug does not take effect when chemotherapy is given to a cancer patient, the resistance is often developing to other anticancer drugs later, and it can be often observed that there is no therapeutic effect even though complex chemotherapy, in which various types of anticancer drugs with different mechanisms of action are administered at the same time during initial treatment, is given. As a result, the limited range of available anticancer drugs is recognized as an important problem in the chemotherapy of cancers. Accordingly, the selection using suitable therapeutic response markers can lead to a significant progress in the treatment with anticancer drugs. Thus, extensive research on the therapeutic response to individual anticancer drugs according to SNP genotype has recently been continuously carried out.

However, due to the complex interaction of biological response-related factors to a specific drug, the diversity of therapeutic agents and administration routes, and the difficulty in securing massive amounts of samples, there is no remarkable achievement yet.

### SUMMARY OF THE INVENTION

To make up for these weaknesses, the present inventors have determined the drug response of tumors using an in vitro tumor response assay, selected a result from lymphocyte DNA of the same patient by association analysis, and discovered SNP markers for predicting the sensitivity to specific anticancer targeted drugs based on human genetic information and population genetic analysis (Korean Patent Application No. 10-2009-0130540), thus completing the present invention.

Accordingly, an object of the present invention is to provide a single nucleotide polymorphism (SNP) marker for predicting the sensitivity to an anticancer targeted drug.

Another object of the present invention is to provide a method for predicting the sensitivity to a specific anticancer targeted drug.

To achieve the above objects, the present invention provides a polynucleotide, or a complementary polynucleotide thereof, for predicting sensitivity to one or more anticancer targeted drugs, selected from polynucleotides comprising at least 8 contiguous nucleotides including each nucleotide at a specific position of the following SEQ ID NOS: 1 to 11.

Preferably, the nucleotide at a specific position may be a nucleotide at position 201 of one of SEQ ID NOS: 1, 7, and 10, a nucleotide at position 251 of SEQ ID NO: 3, a nucleotide at position 301 of one of SEQ ID NOS: 5 and 11, a nucleotide at position 401 of one of SEQ ID NOS: 4, 6, 8, 9, and a nucleotide at position 406 of SEQ ID NO: 2.

Preferably, the at least 8 contiguous nucleotides may be 8 to 100 contiguous nucleotides.

Moreover, the present invention provides a primer or probe for predicting sensitivity to anticancer targeted drugs, the primer comprising the polynucleotide or complementary polynucleotide thereof.

Furthermore, the present invention provides a microarray for predicting sensitivity to anticancer targeted drugs, the microarray comprising the polynucleotide, a polypeptide encoded by the same, or a cDNA thereof.

In addition, the present invention provides a kit for predicting sensitivity to anticancer targeted drugs, the kit comprising the microarray.

Additionally, the present invention provides a method for predicting sensitivity to anticancer targeted drugs, the method comprising the steps of (1) isolating nucleic acid molecules from a subject; and (2) determining the type of a nucleotide at the position of SNP of a polynucleotide comprising SEQ ID NOS: 1 to 11 from the isolated nucleic acid molecules.

In step (2) of the prediction method, when the nucleotide at the position of the SNP, corresponding to the nucleotide at position 201 of SEQ ID NO: 1, is determined as A, it may be predicted that the sensitivity to bevacizumab, an anticancer targeted drug, is high.

In step (2) of the prediction method, when the nucleotide at the position of the SNP, corresponding to the nucleotide at position 406 of SEQ ID NO: 2, is determined as T, when the nucleotide at the position of the SNP, corresponding to the nucleotide at position 251 of SEQ ID NO: 3, is determined as C, or when the nucleotide at the position of the SNP, corresponding to the nucleotide at position 401 of SEQ ID NO: 4, is determined as T, it may be predicted that the sensitivity to at least one of bevacizumab and FOLFIRI (5-FU+leucovorin+irinotecan), anticancer targeted drugs, is high.

In step (2) of the prediction method, when the nucleotide at the position of the SNP, corresponding to the nucleotide at position 301 of SEQ ID NO: 5, is determined as G, it may be predicted that the sensitivity to at least one of bevacizumab and FOLFOX (5-FU+leucovorin+oxaliplatin), anticancer targeted drugs, is high.

In step (2) of the prediction method, when the nucleotide at the position of the SNP, corresponding to the nucleotide at position 401 of SEQ ID NO: 6, is determined as G, it may be predicted that the sensitivity to cetuximab, an anticancer targeted drug, is high.

In step (2) of the prediction method, when the nucleotide at the position of the SNP, corresponding to the nucleotide at position 201 of SEQ ID NO: 7, is determined as A, when the nucleotide at the position of the SNP, corresponding to the nucleotide at position 401 of SEQ ID NO: 8, is determined as G, or when the nucleotide at the position of the SNP, corresponding to the nucleotide at position 401 of SEQ ID NO: 9, is determined as C, it may be predicted that the sensitivity to at least one of cetuximab and FOLFIRI (5-FU+leucovorin+irinotecan), anticancer targeted drugs, is high.

In step (2) of the prediction method, when the nucleotide at the position of the SNP, corresponding to the nucleotide at position 201 of SEQ ID NO: 10, is determined as G, or when the nucleotide at the position of the SNP, corresponding to the nucleotide at position 301 of SEQ ID NO: 11, is determined as T, it may be predicted that the sensitivity to at least one of cetuximab and FOLFOX (5-FU+leucovorin+oxaliplatin), anticancer targeted drugs, is high.

The present invention enables to predict useful drugs against tumors that are resistant to drugs using a small amount of blood sample from a patient, enables to select the most suitable anticancer targeted drug over the entire therapeutic regimen of a metastatic or recurrent cancer patient, and has developed an appropriate diagnosis tool.

Moreover, the present invention enables to newly discover SNP markers that can predict the response to anticancer drugs and common therapeutic agents using drugs including new drugs continuously in the future and can extend the application range.

### BRIEF DESCRIPTION OF THE DRAWINGS

FIG. 1 shows 15 candidate SNPs of selected 15 types of genes sensitive to 6 types of anticancer targeted agents, in which TCF19 rs2073721 and MLPH rs3817362 deviate from Hardy-Weinberg equilibrium in a normal population and thus are excluded from the candidate SNPs.

FIG. 2 shows the results of association analysis on bevacizumab-sensitive SNP genotypes in colorectal cancer patients whose treatment with the corresponding drugs has been terminated in clinics. Metastatic and recurrent cancer patients treated with bevacizumab, an anticancer targeted drug, and carrying homozygous or heterozygous substitution alleles of ITGA3 rs2230392 SNP exhibit greater sensitivity and longer progression-free survival and overall survival than those with homozygous reference alleles.

FIG. 3 shows the results of association analysis on cetuximab-sensitive SNP genotypes on colorectal cancer patients whose treatment with the corresponding drugs has been terminated in clinics. Metastatic and recurrent cancer patients treated with cetuximab, an anticancer targeted drug, and carrying homozygous reference alleles of ISX rs361863 SNP exhibit greater sensitivity and longer progression-free survival than those with homozygous or heterozygous substitution alleles.

### DETAILED DESCRIPTION

The present invention relates to single nucleotide polymorphism (SNP) markers for predicting the sensitivity to anticancer targeted drugs.

The SNPs may be screened by the method disclosed in Korean Patent Application No. 10-2009-0130540. More specifically, the SNPs for predicting the sensitivity to anticancer targeted drugs may be screened by a method comprising the steps of:

(1) selecting candidate SNP genotypes for predicting the sensitivity to anticancer targeted drugs by performing association analysis on the result of an in vitro chemosensitivity assay using patients tumor tissues and the result of a SNP analysis on the same patients; and

(2) selecting SNP genotypes from the selected genotypes having a nominal P value is 1% or less based on the frequency of alleles in Asian genotypes, genotypes located in the linkage disequilibrium block, haplotype tagging SNPs (htSNPs), functional SNPs, and Hardy-Weinberg equilibrium P values in a normal population.

In the above method, the in vitro chemosensitivity assay of step (1) may be performed by the method disclosed in Korean Patent Application No. 10-2008-0115296. The in vitro chemosensitivity assay may comprise the steps of:

(i) preparing a primary anti-cancer drug reaction plate and a secondary anti-cancer drug reaction plate;

(ii) injecting a tumor tissue sample prepared from a subject into reaction wells of the primary anti-cancer drug reaction plate and reaction wells of the secondary anti-cancer drug reaction plate and culturing;

(iii) treating and reacting primary anti-cancer drug candidates with the reaction wells of the primary reaction plate and the reaction wells of the secondary reaction plate;

(iv) measuring and analyzing the sensitivity of the primary anti-cancer drug candidates treated with the reaction wells of the primary anti-cancer drug reaction plate and treating and reacting secondary anti-cancer drug candidates, which have a combination different from that of the primary anti-cancer drug, with the reaction wells of the secondary anti-cancer drug reaction plate; and

(v) measuring and analyzing the sensitivity of the secondary anti-cancer drug candidates applied to the reaction wells of the secondary anti-cancer drug reaction plate, and the method can screen even the sensitivity of new drugs without analysis on clinical outcomes over the years.

The assay in step (2) is intended for haplotype tagging SNPs (htSNPs), functional SNPs, and SNPs with Hardy-Weinberg equilibrium P values greater than 0.01 when frequency of minor alleles similar to that of the known Korean database is greater than 5% in the Japanese and Chinese database (there are no recombinants in the Japanese database with linkage disequilibrium (LD) block; WGA Viewer, Ge D, Zhang K, Need AC, et al. GenomeRes 2008; 18:640-643) as a result of the association analysis on the high-density and high-throughput SNP assay (using the Affymetrix SNP Array 6.0) and the in vitro chemosensitivity assay. The reason that the frequency of minor alleles is defined as 5% is to calculate a minimum value of statistical significance that can reduce the number of targeted samples without missing rare mutations.

The linkage disequilibrium (LD) block is caused when a specific genomic region is short to the extent that no crossover has occurred over the generations or when genes are concentrated. Accordingly, the genomic information present in this region is substantially the same and almost preserved and, similarly to this, the haplotype tagging SNPs (htSNPs) refer to a minimum set of SNPs that can identify specific haplotypes from the total haplotypes and represent adjacent SNPs. Accordingly, the LD block SNPs and htSNPs can eliminate the effort of repeatedly assaying SNPs with similar characteristics, thereby reducing the time and expense.

The Hardy-Weinberg equilibrium refers to genetic stability in a random mating population and is usually represented as a P value. When this value is less than 0.01, it represents a deviation from genetic equilibrium caused by inbreeding, genetic drift, mutation, selection, and errors in genetic analysis or selection of population, and thus false positive SNPs resulting from these results can be excluded.

Candidate SNP markers sensitive to 11 anticancer targeted drugs of 11 types of genes discovered in the assay in step 2 are shown in Table 1 of Example 2.

Preferably, the screening method may further comprise the step of performing association analysis on the SNP genotypes selected in step (2) with existing clinical outcomes. The clinical association can be investigated for subjects whose treatment with anticancer targeted drugs has been terminated.

In an embodiment of the present invention, the prediction of clinical outcomes of the corresponding patients may be based on the NCCN surveillance guidelines (www.nccn.org), and the determination of chemosensitivity to anticancer drugs may be based on the RECIST criteria (Therasse et al., J Natl Cancer Inst 2000; 92:205-16). The observation period for the corresponding patients is an average of 13 months (in a range of 1 to 39 month), which is considered as a sufficient period for the determination of the results considering that these drugs are administered to recurrent and metastatic cancer patients. Through this step, the difference in an in vivo drug metabolism environment of the in vitro chemosensitivity assay can be excluded, and it is possible to enter directly into a clinical test.

The screening method may further comprise a cell biological assay. If the result of the cell biological assay is the same as the result of the clinical association analysis, the sensitivity of the corresponding SNP to the targeted drug is clearly proven, and even if the results do not coincide exactly, it cannot be said that there is no sensitivity of the corresponding SNP. This is because all sensitive SNPs do not exhibit 100% sensitivity. This includes genetic transformation, cell viability and cytotoxicity assays, and apoptosis assay by caspase-3 Western blot and flow cytometric analysis. As a result, it is possible to develop a biological mechanism for the SNP genotypes of the present invention and apply to targeted candidate materials in the development of new drugs.

In one aspect, the cell biological assay may comprise the steps of transforming tumor cells with wild-type genes or mutant genes; treating the tumor cells with a particular anticancer drug; and measuring the cell viability of the tumor cells.

The SNPs of the present invention will be described in detail below.

A SNP for predicting the sensitivity to bevacizumab, an anticancer targeted drug, comprises an rs2230392 (SNP ID) polynucleotide (SEQ ID NO: 1, Chromosome 17), which is part of ITGA3 (integrin alpha 3) gene, containing a nucleotide at position 201 of the nucleotide sequence and at least 8 contiguous nucleotides, or a complementary polynucleotide thereof. Individuals carrying substitution alleles exhibit greater response to bevacizumab, an anticancer targeted drug, than those with reference alleles.

Bevacizumab is an antibody against VEGF.

A SNP for predicting the sensitivity to bevacizumab and FOLFIRI (5-FU+leucovorin+irinotecan: a complex drug of anticancer drug 5-FU and irinotecan, and leucovorin is used to increase the activity of 5-FU), anticancer targeted drugs, comprises an rs1534443 polynucleotide (SEQ ID NO: 2, Chromosome 6), which is part of UTRN (utrophin) gene, containing a nucleotide at position 406 of the nucleotide sequence and at least 8 contiguous nucleotides, or a complementary polynucleotide thereof. Individuals carrying substitution alleles exhibit greater response to at least one of bevacizumab and FOLFIRI, anticancer targeted drugs, than those with reference alleles.

Another SNP for predicting the sensitivity to bevacizumab and FOLFIRI, anticancer targeted drugs, comprises an rs13321 polynucleotide (SEQ ID NO: 3, Chromosome 9), which is part of TNC (tenascin C) gene, containing a nucleotide at position 251 of the nucleotide sequence and at least 8 contiguous nucleotides, or a complementary polynucleotide thereof. Individuals carrying reference alleles exhibit greater response to at least one of bevacizumab and FOLFIRI, anticancer targeted drugs, than those with substitution alleles.

Still another SNP for predicting the sensitivity to bevacizumab and FOLFIRI, anticancer targeted drugs, comprises an rs1049550 polynucleotide (SEQ ID NO: 4, Chromosome 10), which is part of ANXA11 (annexin A11) gene, containing a nucleotide at position 401 of the nucleotide sequence and at least 8 contiguous nucleotides, or a complementary polynucleotide thereof. Individuals carrying substitution alleles exhibit greater response to at least one of bevacizumab and FOLFIRI, anticancer targeted drugs, than those with reference alleles.

A SNP for predicting the sensitivity to bevacizumab and FOLFOX (5-FU+leucovorin+oxaliplatin: a complex drug of anticancer drug 5-FU and oxaliplatin, and leucovorin is used to increase the activity of 5-FU), anticancer targeted drugs, comprises an rs11247226 polynucleotide (SEQ ID NO: 5, Chromosome 15), which is part of LINS1 (lines homolog 1) gene, containing a nucleotide at position 301 of the nucleotide sequence and at least 8 contiguous nucleotides, or a complementary polynucleotide thereof. Individuals carrying substitution alleles exhibit greater response to at least one of bevacizumab and FOLFOX, anticancer targeted drugs, than those with reference alleles.

A SNP for predicting the sensitivity to cetuximab an anticancer targeted drug, comprises an rs2278108 polynucleotide (SEQ ID NO: 6, Chromosome 6), which is part of ZKSCAN3 (zinc finger with KRAB and SCN domains 3) gene, containing a nucleotide at position 401 of the nucleotide sequence and at least 8 contiguous nucleotides, or a complementary polynucleotide thereof. Individuals carrying substitution alleles exhibit greater response to cetuximab, an anticancer targeted drug, than those with reference alleles.

A SNP for predicting the sensitivity to cetuximab and FOLFIRI (5-FU+leucovorin+irinotecan: a complex drug of anticancer drug 5-FU and irinotecan, and leucovorin is used to increase the activity of 5-FU), anticancer targeted drugs, comprises an rs4932305 polynucleotide (SEQ ID NO: 7, Chromosome 15), which is part of SEMA4B (semaphorin 4B) gene, containing a nucleotide at position 201 of the nucleotide sequence and at least 8 contiguous nucleotides, or a complementary polynucleotide thereof. Individuals carrying substitution alleles exhibit greater response to at least one of cetuximab and FOLFIRI, anticancer targeted drugs, than those with reference alleles.

Another SNP for predicting the sensitivity to cetuximab and FOLFIRI, anticancer targeted drugs, comprises an rs2274159 polynucleotide (SEQ ID NO: 8, Chromosome 9), which is part of DFNB31 (deafness, autosomal recessive 31) gene, containing a nucleotide at position 401 of the nucleotide sequence and at least 8 contiguous nucleotides, or a complementary polynucleotide thereof. Individuals carrying substitution alleles exhibit greater response to at least one of cetuximab and FOLFIRI, anticancer targeted drugs, than those with reference alleles.

Still another SNP for predicting the sensitivity to cetuximab and FOLFIRI, anticancer targeted drugs, comprises an rs361863 polynucleotide (SEQ ID NO: 9, Chromosome 22), which is part of ISX (intestine-specific homeobox) gene, containing a nucleotide at position 401 of the nucleotide sequence and at least 8 contiguous nucleotides, or a complementary polynucleotide thereof. Individuals carrying reference alleles exhibit greater response to at least one of cetuximab and FOLFIRI, anticancer targeted drugs, than those with substitution alleles.

A SNP for predicting the sensitivity to cetuximab and FOLFOX (5-FU+leucovorin+oxaliplatin: a complex drug of anticancer drug 5-FU and oxaliplatin, and leucovorin is used to increase the activity of 5-FU), anticancer targeted drugs, comprises an rs3729740 polynucleotide (SEQ ID NO: 10, Chromosome 5), which is part of LIFR (leukemia inhibitory factor receptor alpha) gene, containing a nucleotide at position 201 of the nucleotide sequence and at least 8 contiguous nucleotides, or a complementary polynucleotide thereof. Individuals carrying reference alleles exhibit greater response to at least one of cetuximab and FOLOFX, anticancer targeted drugs, than those with substitution alleles.

Another SNP for predicting the sensitivity to cetuximab and FOLFOX, anticancer targeted drugs, comprises an rs2306393 polynucleotide (SEQ ID NO: 11, Chromosome 12), which is part of MDM1 (Mdm1 nuclear protein homolog), containing a nucleotide at position 301 of the nucleotide sequence and at least 8 contiguous nucleotides, or a complementary polynucleotide thereof. Individuals carrying reference alleles exhibit greater response to at least one of cetuximab and FOLOFX, anticancer targeted drugs, than those with substitution alleles.

The SNP stands for single nucleotide polymorphism, in which the rs is the abbreviation for reference sequence and the following number represents the accession number that identifies each single nucleotide polymorphism provided by a database called dbSNP.

That is, the present invention provides a polynucleotide, or a complement thereof, for predicting the sensitivity to one or more anticancer targeted drugs, selected from polynucleotides comprising at least 8 contiguous nucleotides including each nucleotide at a specific position of the following SEQ ID NOS: 1 to 11:

1. a nucleotide at position 201 of one of SEQ ID NOS: 1, 7, and 10;

2. a nucleotide at position 251 of SEQ ID NO: 3;

3. a nucleotide at position 301 of one of SEQ ID NOS: 5 and 11;

4. a nucleotide at position 401 of one of SEQ ID NOS: 4, 6, 8, 9; and

5. a nucleotide at position 406 of SEQ ID NO: 2.

The at least 8 contiguous nucleotides may preferably be 8 to 100 contiguous nucleotides.

In the present invention, the prediction of the sensitivity refers to the prediction of therapeutic effects of a specific anticancer drug administered to a patient. It is expected that high sensitivity to the corresponding anticancer targeted drug provides excellent therapeutic effects, while low sensitivity to the corresponding anticancer targeted drug provides low therapeutic effects.

In the present invention, the single nucleotide polymorphism (SNP) is a single base-pair variation present in DNA between individuals and is the most abundant type of DNA sequence polymorphism.

The above-mentioned polynucleotides of SEQ ID NOS: 1 to 11 are polymorphic sequences. The polymorphic sequence refers to a nucleotide sequence containing a polymorphic site at which single-nucleotide polymorphism (SNP) occurs. The polynucleotide may be DNA or RNA.

Moreover, the above-mentioned polynucleotides of SEQ ID NOS: 1 to 11 are allele-specific. The allele-specific polynucleotide refers to a polynucleotide specifically hybridized with each allele.

That is, the allele-specific polynucleotide has the ability that distinguishes nucleotides of polymorphic sites within the polymorphic sequences of SEQ ID NOS: 1-11 and specifically hybridizes with each of the nucleotides. The hybridization is usually performed under stringent conditions such as at a salt concentration of no more than 1 M and a temperature of 25° C. or higher. For example, conditions of 5×SSPE (750 mM NaCl, 50 mM Na Phosphate, 5 mM EDTA, pH 7.4) and 25 to 30° C. may be suitable for allele-specific probe hybridization.

In the present invention, the allele-specific polynucleotide may be a primer. As used herein, the primer refers to a single stranded oligonucleotide that acts as a starting point of template-directed DNA synthesis under appropriate conditions in an appropriate solution (e.g., dNTP or dUTP and DNA, RNA polymerase or reverse transcriptase) and at an appropriate temperature. The appropriate length of the primer may vary according to the purpose of use, generally 15 to 30 nucleotides. Generally, a shorter primer molecule requires a lower temperature to form a stable hybrid with a template. Preferably, the 3′ end of the primer is aligned with a polymorphic site of SEQ ID NOS: 1 to 11. The primer is hybridized with a target DNA containing a polymorphic site and starts an allelic amplification in which the primer exhibits complete homology with the target DNA. The primer is used in pair with a second primer hybridizing with an opposite strand. Amplified products are obtained by amplification using the two primers, which means that there is a specific allelic form.

In the present invention, the allele-specific polynucleotide may be a probe. As used herein, the probe refers to a hybridization probe, that is, an oligonucleotide capable of sequence-specifically binding with a complementary strand of a nucleic acid. The probe according to the present invention is an allele-specific probe. In this regard, when there are polymorphic sites in nucleic acid fragments derived from two members of the same species, the probe is hybridized with DNA fragments derived from one member but is not hybridized with DNA fragments derived from the other member. In this case, hybridization conditions should be stringent enough to allow hybridization with only one allele by significant difference in hybridization strength between alleles. Preferably, the central portion of the probe (e.g., position 7 for a 15 nucleotide probe, or position 8 or 9 for a 16 nucleotide probe) is aligned with each polymorphic site of the nucleotide sequences of SEQ ID NOS: 1 to 11. As a result, a significant difference in hybridization between alleles may be caused.

Another aspect of the present invention relates to a polynucleotide comprising the SNP for predicting the sensitivity to a specific anticancer targeted drug according to the present invention and a microarray comprising a polypeptide encoded by the same or a cDNA thereof. The microarray according to the present invention may be prepared using the SNP for predicting the sensitivity to an anticancer drug by a common method known in the art.

For example, the polynucleotide may be immobilized on a substrate coated with an active group selected from the group consisting of amino-silane, poly-L-lysine, and aldehyde. Moreover, the substrate may be selected from the group consisting of a silicon wafer, glass, quartz, metal, and plastic. The immobilization of the polynucleotide on the substrate may be performed by micropipetting using a piezoelectric method or by using a spotter in the shape of a pin.

Still another aspect of the present invention relates to a kit for predicting the sensitivity to an anticancer targeted drug, comprising the microarray according to the present invention.

The kit according to the present invention may further comprise a primer set used to isolate and amplify DNA including the corresponding SNP from a subject in addition to the microarray according to the present invention. A suitable primer set may be easily designed by those skilled in the art with reference to the sequences of the present invention.

Yet another aspect of the present invention relates to a method for predicting the sensitivity to an anticancer drug using the SNP for predicting the sensitivity to an anticancer drug according to the present invention.

The method comprises the steps of

(1) isolating nucleic acid molecules from a subject; and

(2) determining the type of a nucleotide at the position of SNP of a polynucleotide comprising SEQ ID NOS: 1 to 11 from the isolated nucleic acid molecules.

For example, DNA is isolated from the patient's tissues, fluids, or cells, amplified by PCT, and subjected to SNP analysis. The SNP analysis may be performed by a common method known in the art. For example, the SNP analysis may be performed using a real time PCR system or by direct determination of the nucleotide sequence of the nucleic acid by a dideoxy method. Otherwise, the SNP analysis may be performed by hybridizing the DNA with a probe containing the sequence of the SNP site or a complementary probe thereof and examining the degree of the hybridization. Moreover, the SNP analysis may be performed by a method selected from the group consisting of allele-specific probe hybridization, allele-specific amplification, sequencing, 5′ nuclease digestion, molecular beacon assay, oligonucleotide ligation assay, size analysis, and single-stranded conformation polymorphism.

In the prediction method, when the nucleotide at the position of the SNP, corresponding to the nucleotide at position 201 of SEQ ID NO: 1, is determined as A (A/A or G/A heterozygous genotype) in step (2), it may be predicted that the sensitivity to bevacizumab, an anticancer targeted drug, is high.

When the nucleotide at the position of the SNP, corresponding to the nucleotide at position 406 of SEQ ID NO: 2, is determined as T (T/T or C/T heterozygous genotype), it may be predicted that the sensitivity to at least one of bevacizumab and FOLFIRI (5-FU+leucovorin+irinotecan), anticancer targeted drugs, is high.

When the nucleotide at the position of the SNP, corresponding to the nucleotide at position 251 of SEQ ID NO: 3, is determined as C (C/C), it may be predicted that the sensitivity to at least one of bevacizumab and FOLFIRI (5-FU+leucovorin+irinotecan), anticancer targeted drugs, is high.

When the nucleotide at the position of the SNP, corresponding to the nucleotide at position 401 of SEQ ID NO: 4, is determined as T (T/T), it may be predicted that the sensitivity to at least one of bevacizumab and FOLFIRI (5-FU+leucovorin+irinotecan), anticancer targeted drugs, is high.

When the nucleotide at the position of the SNP, corresponding to the nucleotide at position 301 of SEQ ID NO: 5, is determined as G (G/G or A/G heterozygous genotype), it may be predicted that the sensitivity to at least one of bevacizumab and FOLFOX (5-FU+leucovorin+oxaliplatin), anticancer targeted drugs, is high.

When the nucleotide at the position of the SNP, corresponding to the nucleotide at position 401 of SEQ ID NO: 6, is determined as G (G/G or C/G heterozygous genotype), it may be predicted that the sensitivity to cetuximab, an anticancer targeted drug, is high.

When the nucleotide at the position of the SNP, corresponding to the nucleotide at position 201 of SEQ ID NO: 7, is determined as A (A/A or G/A heterozygous genotype), it may be predicted that the sensitivity to at least one of cetuximab and FOLFIRI (5-FU+leucovorin+irinotecan), anticancer targeted drugs, is high.

When the nucleotide at the position of the SNP, corresponding to the nucleotide at position 401 of SEQ ID NO: 8, is determined as G (G/G), it may be predicted that the sensitivity to at least one of cetuximab and FOLFIRI (5-FU+leucovorin+irinotecan), anticancer targeted drugs, is high.

When the nucleotide at the position of the SNP, corresponding to the nucleotide at position 401 of SEQ ID NO: 9, is determined as C (C/C), it may be predicted that the sensitivity to at least one of cetuximab and FOLFIRI (5-FU+leucovorin+irinotecan), anticancer targeted drugs, is high.

When the nucleotide at the position of the SNP, corresponding to the nucleotide at position 201 of SEQ ID NO: 10, is determined as G (G/G), it may be predicted that the sensitivity to at least one of cetuximab and FOLFOX (5-FU+leucovorin+oxaliplatin), anticancer targeted drugs, is high.

When the nucleotide at the position of the SNP, corresponding to the nucleotide at position 301 of SEQ ID NO: 11, is determined as T (T/T or T/C heterozygous genotype), it may be predicted that the sensitivity to at least one of cetuximab and FOLFOX (5-FU+leucovorin+oxaliplatin), anticancer targeted drugs, is high.

According to an embodiment of the present invention, as a result of performing clinical association analysis on subjects treated with bevacizumab or cetuximab, the subjects carrying reference alleles such as DFNB31 rs2274159, LIFR rs3729740, and ISX rs361863 and substitution alleles such as ANXA11 rs1049550, ITGA3 rs2230392, and LINS1 rs11247226 exhibited greater response to anticancer drugs or longer progression-free survival (Example 3).

The following Examples are given for illustrating the present invention in more detail, and it will be understood by those skilled in the art to which the present invention pertains that the scope of the present invention is not limited by the following examples.

### Example 1

### Step (1) to Determine SNP Genotypes

Basic data of SNP genotypes sensitive to anticancer targeted drugs were obtained by selecting 56 SNPs having a linkage disequilibrium (R²) value of greater than 0.8 from a total 4,163 SNPs having a −Log(P) value of greater than 3 by performing association analysis on the result of a high-density and high-throughput SNP assay (using the Affymetrix SNP Array 6.0) and the result of an in vitro chemosensitivity assay in 118 colorectal cancer patients.

### Example 2

### Step (2) to Determine SNP Genotypes

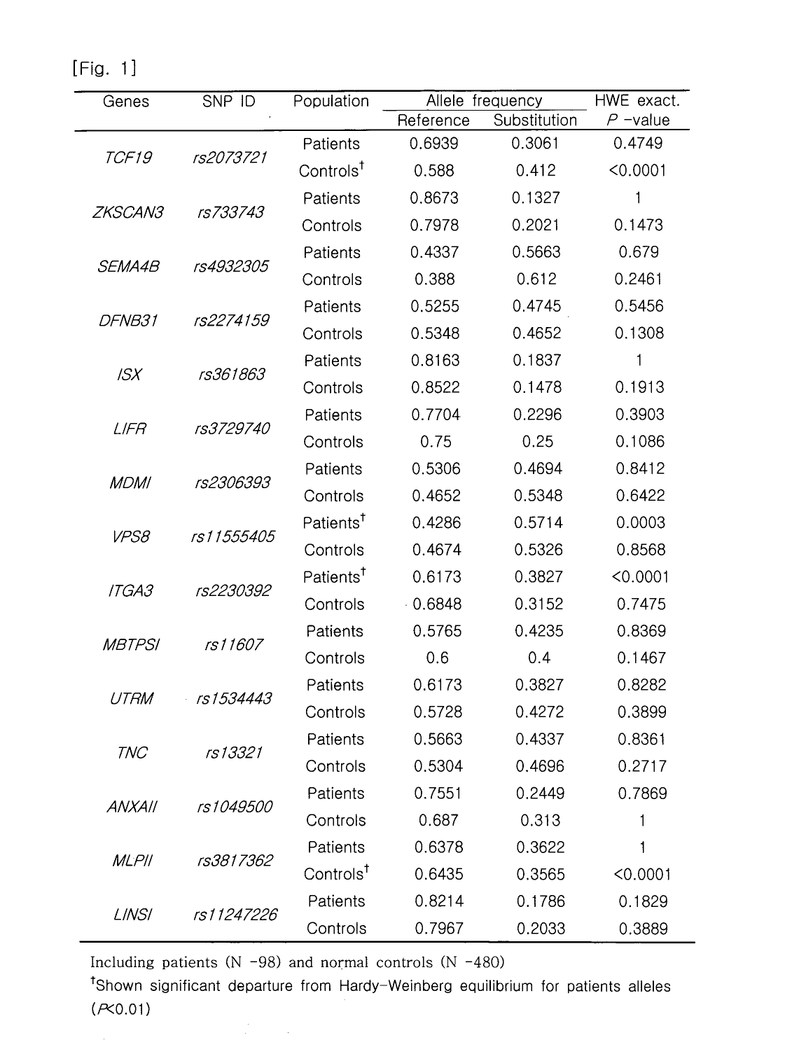

Candidate SNP genotypes exhibiting a frequency of 10% or higher (www.hanpmap.org) in existing Japanese and Chinese analysis data and exhibiting linkage disequilibrium were selected from the SNP genotypes selected in step (1), and further 15 candidate SNPs were discovered by non-synonymous, haplotype-tagging, and functional SNP selection (FIG. 1).

Then, 11 SNPs without significant deviation from Hardy-Weinberg equilibrium (P>0.01) in the same SNP genotype assay using lymphocyte DNA samples in 480 normal populations were selected as candidates for clinical association analysis for the final assay in an Example of step (3).

TABLE 1
Candidate SNPs of 11 genes discovered in step (2)
sensitive to 11 anticancer drugs

| | | | Alleles | | |
|---|---|---|---|---|---|
| | | | Refer- | Substi- | |
| Regimens | Genes | SNP ID | ence | tution | Results |
| Bevacizumab | ITGA3 | rs2230392 | G | A | A > T |
| Bevacizumab + | UTRN | rs1534443 | C | T | S > N |
| FOLFIRI* | | | | | |
| Bevacizumab + | TNC | rs13321 | C | G | E > Q |
| FOLFIRI* | | | | | |
| Bevacizumab + | ANXA11 | rs1049550 | C | T | R > C |
| FOLFIRI* | | | | | |
| Bevacizumab + | LINS1 | rs11247226 | A | G | I > V |
| FOLFOX* | | | | | |
| Cetuximab | ZKSCAN3 | rs733743 | C | G | R > T |
| Cetuximab + | SEMA4B | rs4932305 | G | A | I > M |
| FOLFIRI* | | | | | |
| Cetuximab + | DFNB31 | rs2274159 | G | A | A > V |
| FOLFIRI* | | | | | |
| Cetuximab + | ISX | rs361863 | C | T | S > G |
| FOLFIRI* | | | | | |
| Cetuximab + | LIFR | rs3729740 | G | A | D > N |
| FOLFOX* | | | | | |
| Cetuximab + | MDM1 | rs2306393 | T | C | H > R |
| FOLFOX* | | | | | |

*FOLFOX, FL (5-FU/leucovorin) + irinotecan; FOLFOX, FL (5-FU/leucovorin) + oxaliplatin

### Example 3

### Association Analysis Step Between SNP Genotypes and Existing Clinical Outcomes

Clinical association analysis was performed on 69 subjects treated with bevacizumab and 67 subjects treated with cetuximab, whose progress of treatment with the corresponding anticancer drugs were confirmed, to investigate the clinical association. The prediction of clinical outcomes of the corresponding patients was based on the NCCN surveillance guidelines (www.nccn.org), and the determination of chemosensitivity to anticancer drugs were based on the RECIST criteria (Therasse et al., J Natl Cancer Inst 2000; 92:205-16). The observation period for the corresponding patients was an average of 13 months (in a range of 1 to 39 month), which was considered as a sufficient period for the determination of the results when considering that these drugs were administered to recurrent and metastatic cancer patients.

3-1: Clinical Association Analysis Related to Survival on Subjects Treated with Anticancer Targeted Drugs

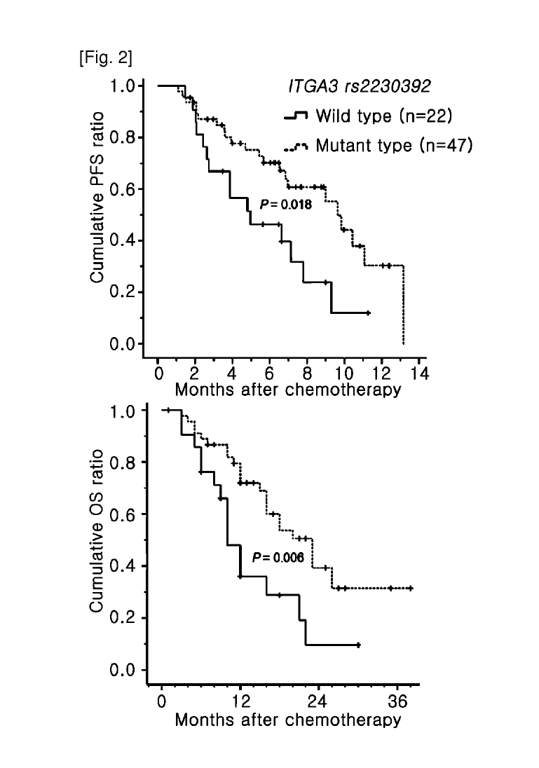

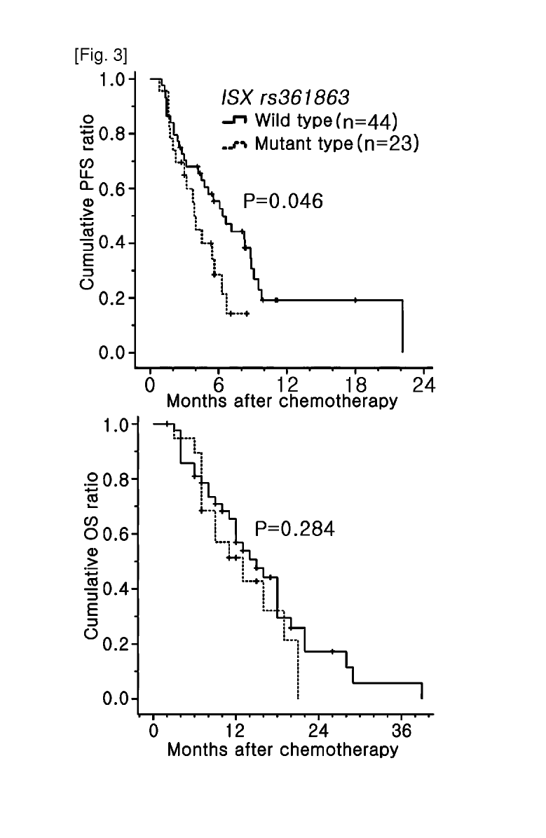

Association analysis was performed on the SNP genotypes sensitive to the individual anticancer drugs obtained in steps (1) and (2) in colorectal cancer patients whose treatment with the corresponding drugs was terminated. First, the association analysis was performed on the candidate SNP genotypes sensitive to the respective drugs in 69 patients treated with bevacizumab and 67 patients treated with cetuximab, including 98 recurrent and metastatic cancer patients with crossover treatment. Patients treated with bevacizumab, an anticancer targeted drug, and carrying homozygous or heterozygous substitution alleles of ITGA3 rs2230392 SNP genotype exhibited significantly longer mean progression-free survival and overall survival than those with homozygous reference alleles (PFS, 8.7±0.7 months vs. 5.7±0.7 months, P=0.018; OS, 22.5±2.1 months vs. 13.4±1.9 months, P=0.006) (FIG. 2). On the contrary, patients treated with cetuximab, an anticancer targeted drug, and carrying homozygous reference alleles of ISX rs361863 SNP genotype exhibited significantly longer mean progression-free survival than those with homozygous or heterozygous substitution alleles (PFS, 8.5±1.2 months vs. 4.4±0.5 months, P=0.046) (FIG. 3).

3-2: Clinical Association Analysis of Drug Response on Subjects Treated with Anticancer Targeted Drugs (Table 2)

As a result of performing clinical association analysis with respect to drug response on the SNP genotypes selected in steps (1) and (2), respectively, in 69 subjects treated with bevacizumab and 67 subjects treated with cetuximab, the subjects treated with bevacizumab and carrying homozygous substitution alleles of ANXA11 rs1049550 and homozygous or heterozygous substitution alleles of LINS1 rs11247226 exhibited a significantly high disease-control response rate (stable disease+partial disease+complete response) (P=0.025, 0.019). Meanwhile, the subjects treated with cetuximab and carrying homozygous reference alleles of DFNB31 rs2274159 and LIFR rs3729740 exhibited significantly high objective response rate (partial disease+complete response) and disease-control response rate (P=0.023, 0.049).

TABLE 2
Sensitivity to anticancer targeted drugs in metastatic
cancer patients based on candidate SNP genotypes

| | | | | No. with disease-control response | | |
|---|---|---|---|---|---|---|
| | | | | (DCR) or objective response (OR)/total | | |
| | | | | patients (%) | | |
| Genes | SNP IDs | Genotypes | Regimens | Types | Responses | P-values |
| ANXA11 | rs1049550 | CC + CT | bevacizumab | DCR | 16/32 (50) | 0.025 |
| | | TT | | | 28/37 (76) | |
| LINS1 | rs11247226 | AA | bevacizumab | DCR | 25/46 (54) | 0.019 |
| | | AG + GG | | | 19/23 (83) | |
| DFNB31 | rs2274159 | GG | cetuximab | OR | 8/17 (47) | 0.023 |
| | | GA + AA | | | 9/50 (18) | |
| LIFR | RS3729740 | GG | cetuximab | DCR | 30/42 (71) | 0.049 |
| | | GA + AA | | | 12/25 (48) | |

1. A polynucleotide, or a complementary polynucleotide thereof, for predicting sensitivity to one or more anticancer targeted drugs, selected from polynucleotides comprising at least 8 contiguous nucleotides including each nucleotide at a specific position of the following SEQ ID NOS: 1 to 11:
1. a nucleotide at position 201 of one of SEQ ID NOS: 1, 7, and 10;
2. a nucleotide at position 251 of SEQ ID NO: 3;
3. a nucleotide at position 301 of one of SEQ ID NOS: 5 and 11;
4. a nucleotide at position 401 of one of SEQ ID NOS: 4, 6, 8, 9; and
5. a nucleotide at position 406 of SEQ ID NO: 2.

2. The polynucleotide of claim 1, wherein the at least 8 contiguous nucleotides are 8 to 100 contiguous nucleotides.

3. A primer for predicting sensitivity to anticancer targeted drugs, the primer comprising the polynucleotide or complementary polynucleotide thereof of claim 1.

4. A probe for predicting sensitivity to anticancer targeted drugs, the probe comprising the polynucleotide or complementary polynucleotide thereof of claim 1.

5. A microarray for predicting sensitivity to anticancer targeted drugs, the microarray comprising the polynucleotide of claim 1, a polypeptide encoded by the same, or a cDNA thereof.

6. A kit for predicting sensitivity to anticancer targeted drugs, the kit comprising the microarray of claim 5.

7. A method for predicting sensitivity to anticancer targeted drugs, the method comprising the steps of:
(1) isolating nucleic acid molecules from a subject; and
(2) determining the type of a nucleotide at the position of SNP of a polynucleotide comprising SEQ ID NOS: 1 to 11 from the isolated nucleic acid molecules.

8. The method of claim 7, wherein in step (2), when the nucleotide at the position of the SNP, corresponding to the nucleotide at position 201 of SEQ ID NO: 1, is determined as A, it is predicted that the sensitivity to bevacizumab, an anticancer targeted drug, is high.

9. The method of claim 7, wherein the in step (2), when the nucleotide at the position of the SNP, corresponding to the nucleotide at position 406 of SEQ ID NO: 2, is determined as T, when the nucleotide at the position of the SNP, corresponding to the nucleotide at position 251 of SEQ ID NO: 3, is determined as C, or when the nucleotide at the position of the SNP, corresponding to the nucleotide at position 401 of SEQ ID NO: 4, is determined as T, it is predicted that the sensitivity to at least one of bevacizumab and FOLFIRI (5-FU+leucovorin+irinotecan), anticancer targeted drugs, is high.

10. The method of claim 7, wherein in step (2), when the nucleotide at the position of the SNP, corresponding to the nucleotide at position 301 of SEQ ID NO: 5, is determined as G, it is predicted that the sensitivity to at least one of bevacizumab and FOLFOX (5-FU+leucovorin+oxaliplatin), anticancer targeted drugs, is high.

11. The method of claim 7, wherein in step (2), when the nucleotide at the position of the SNP, corresponding to the nucleotide at position 401 of SEQ ID NO: 6, is determined as G, it is predicted that the sensitivity to cetuximab, an anticancer targeted drug, is high.

12. The method of claim 7, wherein in step (2), when the nucleotide at the position of the SNP, corresponding to the nucleotide at position 201 of SEQ ID NO: 7, is determined as A, when the nucleotide at the position of the SNP, corresponding to the nucleotide at position 401 of SEQ ID NO: 8, is determined as G, or when the nucleotide at the position of the SNP, corresponding to the nucleotide at position 401 of SEQ ID NO: 9, is determined as C, it is predicted that the sensitivity to at least one of cetuximab and FOLFIRI (5-FU+leucovorin+irinotecan), anticancer targeted drugs, is high.

13. The method of claim 7, wherein in step (2), when the nucleotide at the position of the SNP, corresponding to the nucleotide at position 201 of SEQ ID NO: 10, is determined as G, or when the nucleotide at the position of the SNP, corresponding to the nucleotide at position 301 of SEQ ID NO: 11, is determined as T, it is predicted that the sensitivity to at least one of cetuximab and FOLFOX (5-FU+leucovorin+oxaliplatin), anticancer targeted drugs, is high.


SEQUENCE LISTING (11 sequences)

SEQ ID NO: 1 - DNA, 401 bp, Homo sapiens
gatggagagaaagggggaaagggaggcagaggtggggtggggaggtagagacccctcacccagaataggaggaggaggagaaggccagaaagccaactagaatagtgtgcatcccctccttccccatgacatcacccccgccaggccccatatgccactctctgcctccctgacccttgtggcagatggagctgctcatcrcctttgaggtcatcggggtgaccctgcacacaagggaccttcaggtgcagctgcagctctccacgtgagtgacctcgaaaagccagtctgggtcagggctgaggtatcttggatcaccttcctaacccctgcatttcctcccaaggtcgagtcaccaggacaacctgtggcccatgatcctcactctgctggtggactat

SEQ ID NO: 2 - DNA, 1351 bp, Homo sapiens
aaacctgatcatatcacaacagtataaatgcaagaaaagctctatgctttgtagttagccctgaatggttgtttgacagatacagaattttttaaacctcattctctcatagtttaggataaacaaaagggattcaaacagatctggtctacctctgggttgtggcgtttccatgacaagcttttacactatgcacctactttcattcgggaaacggtcttattgatctcttctacatccccaacagtgacaatgttggacttcagcatctggtcgattaataccagccagtcggctagttctgttatagttttatcaagatcagcaggaattgaaatttccgaagtacgatgagactgaacaggaatatctgacgcagatgatactagcaccaccttttggacaytaggatgagctgaaatgaaaaatgtagcaaaagagaaattaatcaacaggatgttttaattatggtggtgtcatggtgacacgtgtatgtgtatttaaataaaatttagtttaagtaaaaaaaattctaagaggttattttttcatactgaaatggctatggtattacctgtaatatccatatgtctttggtagctatcaagccaacaaaacagttttgcaacattctataacataatcagcactgacactgagaaaaattaaaagtacaaatgtgggagaactggaagtattattttattttggcaagatgattttaattataaagccaacttattcataattttttatttttttcatttaactttataatgtgagcatattttattcttagtaaacacaattatgaatacctatgtaataacctatacccatattcacttttatttaacccttggatatttagattgctcctgaaattttattttaattttttaaaaaacttgcaaagaatatcttttcatgtagagtattttgcacatttctgatctctatcttagcatagatttcccaagtagaattactgagtcagaaaatacaaaaaaaagtatgtatttaatattcattgccaaattgattggttttgatacaataagtagttgaattagtaattaaaactatctgtaacctacttcaggtatgatatattagatgaggcttaaataattttctaaaatgtatagccaatctgtaaattcagcacaatcctaataaaaatcccaacaggcttgaatgtaacaaaatcattaattcaaatttctggttataaaattaatattgaagagaaaaggacaaaaaacagtcaaattattttttgatagaggaatatgccctcccagatattaagttatattatttagctaataattattaactcagtatatta

SEQ ID NO: 3 - DNA, 501 bp, Homo sapiens
gcccatttctatgagtttttaattgtttgcatcacattattgcacctggaaattccccaggcattctagtccatagagttaagctaagaattaagctccagctcagtcctcctgcactgagagcatcaccatttctccttattccacagttggactcctgtaccccttccccaaggactgctcccaagcaatgctgaatggagacacgacctctggcctctacaccatttatctgaatggtgataaggctsaggcgctggaagtcttctgtgacatgacctctgatgggggtggatggattgtgagtaccaccagggactgcagagccctcaggcctaggggaagagggagagggcaaagcaggggaggagaagagggtgatctcctcctgatgcactctgacacccccatgtgtgaggccttgcaagatagtgcctgtcacacagaggcactcaggaattctctcttaaacaaaagaatgggaaacacgctggctacttt

SEQ ID NO: 4 - DNA, 801 bp, Homo sapiens
cgacatcttacctgagactgtttgtccgagtagtttggaagccgaggcaccatcactgatgctcccggctttgaccccctgcgagatgccgaggtcctgcggaaggccatgaaaggcttcggtaagagaccctgggtggctcaaatcctactccctgccccctatttcccaggcagtttcactgtggctctgtgggctggggtagctgggggacgctgacctggccccagtggtgatagaaggtggatgtgggggtatggctagcatgcccagaacagttgaggggcccaggccagggaagtctcagctgcagagcatctcatgacccctacccgacccacccctgctgcctcttcctccagggacggatgagcaggccatcattgactgcctggggagtygctccaacaagcagcggcagcagatcctactttccttcaagacggcttacggcaaggcgagctgcggggtgggggcgcgggacagtgaggcgtgtcctgggctcagcacagccctgctctcccctgcttttccttgggtgggcccggatctcccggatggacagtaaggagcaggagggcatttccttctgcccatccagctggtggcatcgttgggaggtgaaggatgccaccgggtgctgaggtttgctgagaggctccagacatctatccctgggttctagcccttgctctgccacatctcactgaggttgtggccacatcattttccttcccctgccctcttcatgaccaccctggggggtttagctgatgtaagagctttgcagggaaaggctgt

SEQ ID NO: 5 - DNA, 601 bp, Homo sapiens
gcagtcagcatcccctttaacttgagagacttagtgattaagtggaaacagttgctttctcttgtaataagcatatagcagttagaatttttaaattatttttcctattttaggtttcactgaagaaacttcttagagattcattgcacttctgagatttaatgtttacaacttggagttgtcgaccttcttataagatacattttggaagtcaaaatgaaagttttctgtgaagttttagaagagttatacaagaaggtacttcttggagccacacttgaaaatgacagccatgattacrtcttttatctcaacccagcagtttcagatcaagattgttctacagccacctccttagaatgggcaaacacctgtggtatccagggcaggcatcagcccatctctgttggtgtggctcccattgctgtagcacctgtgtgtttgaagaccaactctcagatgagcggttccagagaagtaatgctccttcagttaacagtgatcaaagtgatgacaacccggatattgtctgtcaaaaccgagttccatgcaaaggagcagtacagagatgtaattaaaattctcttagaatcagccaaag

SEQ ID NO: 6 - DNA, 801 bp, Homo sapiens
ttctacctgcggcgccggctcatccagctgcctctccaaatactccaatagcaccaccacctcctccccgctctctggatgctgctcccgcacccagctctgcagattccccggcaggatggtcaggaactgctccagcaccagcagctccaggatctgctccttgctgtgcatctcaggctgcagccactgtcggcagagctctcggagccgactcagcgcctcgcgggggcctgcagcctccgggtagcggaagcctcggaagcgctcgcgggagccctcagaacccagatctgggctactgggaaaaccggcttcttcttcctccacctttatgaccagaagctccatctggtcttctgtagactgggcatccagggctgtgctttcacttaattctstagccatcctaggccaaggcagtactcaggcctcactctagcttccagcgctatttctgcagaagatccctgaaaggtgggaaatcattattaaccagtaaacttgcaagtagtcccttctctcctgcccaggagcccagagacatctctggggaactgctgcagaaatttctccatgttcagtggttctgcagcttaatcctctacaccaccccaaacctgaatcccatagtaaggttcccaaaggatgtcctggagtgatcctggacacccccaagcccctgactaatcaggaaaaaaactgaaattggtgaggatccagggcacttcctggactctctaggaagctcctctcagtgacttgctcagagttatcccattaccaaccaggtctgtggtt

SEQ ID NO: 7 - DNA, 401 bp, Homo sapiens
cgactgtccaggttcaaacccttcggcaccgtgtcctggctgaggaggacggcgtccctgacacctgaccccttctgtcccacacacctcacctgcctgcagagttgccttctcctgcacgcctctctctttctcaccaagcctgctcctcattccctggagtgcccctgagcccatgtgtccaccctcctgcagaacatrgagaacttcaccctggcaagggacgagaaggggaatgtcctcctggaagatggcaagggccgttgtcccttcgacccgaatttcaagtccactgccctggtggttggtgagtgttgagggcaggatggcttcattgaggttccatgtaggggcacacagggctagagggctagctttggacaggctgagtccatgccatg

SEQ ID NO: 8 - DNA, 801 bp, Homo sapiens
tctggtggtggcatcaggacttgaatcgaagcctgactccgaagactgtctcagccacctggccatgtcaaaactgagaggagcttaaagattcccaagaccaatctcagcctcagcccctagtgtcagtgcaaaggcagagctgaaactgggcctccagattcctaatccagtgctcctccaggacgtccaaatccctccagttatagggagctcactacctccctttgtggtcacttgccaagctgagccctggtcccgggacctcccattctgtgcctggggcccgtgttcctctcagccctctgcatcctcctcctcctggctcaccggtttgttggccccatctgtgggcctctcgttccgaggcagctccttgctactcctgctcttggtggacrccgactgccttcctcggcctggggcgctggcctctgcctcgccagcatccacaccactgtcctcgcttagagtctgcccgctgtccgagaggtgggagagcgtagaggctgctggagaggggaaaaggaagagagaagcagcttatatagctggggtcagggagggctgcctggaggcagtgacggagatggaggctggaaggctgggggaattccccaggcaaagatgggggaaggaatgaagagatcgtggggccagagggcaggtccactccctgccaccctctccacctactcaagacctgtcctttcaaccagacctggtcttgaaggctgtgacttccaggacatctgcctggatctcccagttcccagccactggcataatgataaaacagag

SEQ ID NO: 9 - DNA, 801 bp, Homo sapiens
cggggcctggttacaataacaagccccaggtccctgcattctgcaactaactgagccttggtgtctgaagctgaaaagcacagttttgggttagattgaaaggtatatcaggaaaccagcccagagccaggtcttgctctgaattttagagtcctctatccagttaatatggacggagaagctttttctctgcctgagcccagctgggtcttggagaccaggaaacagaaagaaatgatcagaagacacttaccctggggctggtcctttggaggcttttctagcccagagcctgaggccgcagcttctccggggccctcttcacctggcccttctggtctgtccatatcactcctcctggcaggcctctttaggatcgcctcaatggagaaggacaggcycagcttcttcggggcctcacagcaccccaagctatttctctccatacccctgcagagagcagggcccacctcagcacacattgaggggctcaggggtgactctgtgtaggccaaggagggtgccagtggagagtggcgtacttcctcccctgaacctcctggggtcaagagggcgcgtggaggtgaagaacagagagcctccagttagatgatccaaacaggatttcgtgtccacagggacttccatgggcaggtcacagcagaagagctggaaggagtggtatcctgtgcccacggctcagcccttaccagtttgacatcaaaggagtcaccatggctgaattggggttcaatcttccagagaagggtcttctcggacacagttgcctgcacaatcacc

SEQ ID NO: 10 - DNA, 401 bp, Homo sapiens
gtgctggaattataagcgtgagtcaccacgcctggccaaccttgttattgttaatatagtgtccagtttaaacccaattaccttgcctagtcacagtcacgttttgcagggttttataacaatttgtctgtttttcatagcctttacccattaatgaagctaatggaaaaatactttcctacaatgtatcgtgttcatcaratgaggaaacacagtccctttctgaaatccctgatcctcagcacaaagcagagatacgacttgataagaatgactacatcatcagcgtagtggctaaaaattctgtgggctcatcaccaccttccaaaatagcgagtatggaaattccaaatggtgagtgcttgagatgatttagtatataagaacgggaaactaaagtg

SEQ ID NO: 11 - DNA, 601 bp, Homo sapiens
attataggacgtaaatgaaaataatcctaactacttcctcccaaagtaggccagaattcaagtattcaattaccaatcactagtgacttaaatacctgagtgaatcaaattattcaccatacagcagtgcctcctttgtgacattgaaataaaatctgcaatttattagtcataaaatcaaaagagtaaaccactatatttagtgacaaaatacatatcaaccatgtaaataacgcttctctatactgagtaatttaaaactctcacccttctttcatcttatctgccttagatttctcaygtacatccaacttctcttgctctcccataaaagcctgcttgcccgttttcctgtccccttcctcttcattgtcgtcctcctcctctttctccccgggctgtttctgtacgtcttcacttgtgttctctgtatcccaagccagccgccttctaacgggtatggtgggagctgacacacccaatttctccgtggtatttttctggggctgttcttccacgatatttcctttttcttctggttctgtagaaggacatttctgcaaagtcttatggcttgtaggatctctgcgtaacaaaatca